(19) United States
(12) Patent Application Publication
(10) Pub. No.: US 2025/0259428 A1
(43) Pub. Date: Aug. 14, 2025

(54) METHODS AND SYSTEMS FOR FEDERATED LEARNING OF A MACHINE LEARNED MODEL

(71) Applicant: Siemens Healthineers AG, Forchheim (DE)

(72) Inventors: Varghese KOLLERATHU, Bengaluru (IN); Vineet VINAY BHOMBORE, Bengaluru (IN); Matthias WOLF, Coatesville, PA (US)

(73) Assignee: Siemens Healthineers AG, Forchheim (DE)

(21) Appl. No.: 19/048,085

(22) Filed: Feb. 7, 2025

(30) Foreign Application Priority Data
Feb. 9, 2024 (DE) 10 2024 201 190.8

(51) Int. Cl. G06V 10/776; G06T 7/00; G06V 10/774; G06V 10/778; G16H 30/40

(52) U.S. Cl. CPC G06V 10/776; G06T 7/0012; G06V 10/774; G06V 10/778; G16H 30/40; G06T 2207/20081; G06T 2207/30004; G06V 2201/03

(57) ABSTRACT

A computer-implemented method comprises: receiving, from a first local site remote from a model aggregator device, a local update of a machine learned model and a parameterization of local data, wherein the local update was generated at the first local site based on the local data; generating a synthetic representation of the local data based on the parameterization using a generative AI function; evaluating the local update using the synthetic representation to obtain an evaluation result indicative of the performance of the local update; and updating the machine learned model based on the evaluation result and the local update.

### CROSS-REFERENCE TO RELATED APPLICATION(S)

The present application claims priority under 35 U.S.C. § 119 to German Patent Application No. 10 2024 201 190.8, filed Feb. 9, 2024, the entire contents of which is incorporated herein by reference.

### FIELD

Embodiments of the present invention relate to systems and methods for providing an updated machine learned model in a distributed environment. In particular, embodiments of the present invention relate to the federated learning of a machine learned model so as to provide an updated machine learned model. In particular, embodiments of the present invention relate to the usage of updated machine learned models for medical data processing, and, in particular, for medical image data processing.

### BACKGROUND

Machine learning methods and algorithms are applied widely to generate insights and/or (predictive) computational models from data. Typically, data is brought to a processing unit (such as a cloud) which can run such methods and algorithms to train models or generate insights. In this regard, machine learning methods have proven very versatile in various fields of application. They are used, for instance, to support decision making in autonomous driving. Likewise, machine learning methods are relied on for providing medical diagnoses by automated systems processing physiological measurements such as medical images.

However, due to data privacy regulations, it is often not possible to bring data to an external processing unit which may execute machine learning methods and algorithms but whose ownership deviates from the one of the data. Often, under such circumstances, the data always has to stay on-site with the data owner. This situation in many cases arises in the healthcare business where the inherent sensitivity of patient related data raises important patient privacy concerns.

One way of addressing this problem is to implement a distributed or federated learning scheme. Here, a central machine learned model hosted at a model aggregator device (such as a central server unit) may be improved based on the usage reported by many client units respectively sitting at local sites. Thereby, a readily trained, functioning, and deployable central machine learned model is distributed to the local sites and executed locally. Each client randomly, periodically, or on command may send a local update to the central server unit. The local update may summarize a local change to the machine learned model based on the local data gathered by the client. The model aggregator device may use the local updates to improve the machine learned model. In turn, the model aggregator device may then upload, to the clients, a modified machine learned model that implements the learning modification based on actual usage reported by the client. This enables the clients to collaboratively learn and improve a shared machine learned model while their local and potentially classified data is not distributed outside of the client units.

One issue in this regard is that the local updates have to be evaluated or tested at the model aggregator device in order to determine if they constitute an improvement. To this end, the model aggregator device needs to have test data on the basis of which modifications of the machine learned model are tested. Typically, this test data is a fixed set that has been obtained in compliance with data privacy regulations. Centrally maintaining such a fixed set has several disadvantages. To begin with, such test data is difficult to get and therefore the number of data instances is limited. Moreover, there is no guarantee that the centrally hosted test data is representative of the real-world scenarios and, more importantly, such a fixed data is not resilient to concept drift which may occur in the field.

As an alternative, it has been proposed to use a decentralized approach for evaluating the models (c.f., US 2021/0 097 439 A1, for instance). Here, an aggregated model is sent to all clients to perform evaluation on the local test data. Each client downloads the models, performs inference, and then uploads evaluation results. The central server aggregates the evaluation performance across sites and then decides to keep the model or to discard it. This brings about the advantage that the models are tested on real-world data and the chance of over-fitting is reduced as the test data changes as a function of time. On the downside, however, this approach also suffers from disadvantages. Firstly, the clients need to reserve computational resources not only for training, but also for testing. This might reduce the turnaround times both in model update cycles as well as in the actual usage of the models at the clients. Secondly, this necessarily leads to increased data traffic as the clients need to download the models and upload the results back to the model aggregator device. Thirdly, the local data conceptually will change over time. While this reduces the effect of overfitting, it may become difficult to compare current models with historical models.

### SUMMARY

It is therefore an object of embodiments of the present invention to provide improved methods and systems for federated learning of a machine learned function. In particular, it is an object of embodiments of the present invention to provide systems and methods which allow for a more efficient evaluation of machine learned functions in a distributed environment.

This object is solved by a method for federated learning of a machine learned model, a system for federated learning of a machine learned model corresponding computer-program products, and computer-readable storage media according to the main claims. Alternative and/or preferred embodiments are object of the dependent claims.

In the following, the technical solution according to the present invention is described with respect to the claimed apparatuses as well as with respect to the claimed methods. Features, advantages, or alternative embodiments described herein can likewise be assigned to other claimed objects and vice versa. In other words, claims addressing the inventive method can be improved by features described or claimed with respect to the apparatuses. In this case, e.g., functional features of the method are embodied by objective units or elements of the apparatus.

The technical solution will be described both with regard to methods and systems for providing an updated machine learned function and also with regard to methods and systems for providing training or test data for updating a machine learned system. Features and alternate forms of embodiments of data structures and/or functions for methods and systems for providing machine learned functions can be transferred to analogous data structures and/or functions for methods and systems for providing training or test data. Analogous data structures can, in particular, be identified by using the prefix “training”. Furthermore, the prediction functions used in methods and system for providing information can, in particular, have been adjusted and/or trained and/or provided by methods and systems for adjustment of prediction functions.

According to an aspect, a computer-implemented method for federated learning of a machine learned model in a model aggregator device is provided. The method comprises a plurality of steps. One step is directed to receive, at the model aggregator device, from a local site remote from the model aggregator device, a local update of the machine learned model and a log file, wherein the local update was generated (or provided) at the local site based on local data and the log file comprises a parameterization of the local data. Another step is directed to generate, at the model aggregator device, a synthetic representation of the local data based on the parameterization using a generative AI function. Another step is directed to evaluate, at the model aggregator device, the local update using the synthetic representation so as to obtain an evaluation result indicative of the performance of the model update. Another step is directed to update, at the model aggregator device, the machine learned model based on the evaluation result and the local update.

According to a further aspect, a computer-implemented method for federated learning of a machine learned model in a model aggregator device is provided. The method comprises a plurality of steps. One step is directed to receive, at the model aggregator device, from a first local site remote from the model aggregator device, a first local update of the machine learned model and a log file, wherein the first local update was generated (or provided) at the local site based on local data and the log file comprises a parameterization of the local data. Another step is directed to generate, at the model aggregator device, a synthetic representation of the local data based on the parameterization using a generative AI function. Another step is directed to receive, at the model aggregator device, from a second local site remote from the model aggregator device and different from the first local site, a second local update of the machine learned different from the first local update. Another step is directed to evaluate, at the model aggregator device, the second local update using the synthetic representation so as to obtain an evaluation result indicative of the performance of the second model update. Another step is directed to update, at the model aggregator device, the machine learned model based on the evaluation result and the second local update.

The model aggregator device may be central server unit which is configured to administrate the federated learning of the machine learned model. For instance, the model aggregator device may comprise a web server. Further, the model aggregator device may comprise a cloud server or a local server. The model aggregator device may be in data communication with one or more local sites. The model aggregator device may be configured to provide the machine-learned model to the local sites and to obtain updated machine-learned models from the local sites (local updates). The model aggregator device may further be configured to evaluate the local updates and to decide about an integration of features of the local updates in the machine-learned model based on the evaluating step. The model aggregator device may comprise an interface unit for facilitating data communication with the local sites, for instance via internet connection.

The local site may be conceived as a client or client unit in the federated learning network administrated by the model aggregator device. The local sites may, in particular, comprise to a local computer network comprising one or more computing units. The local sites may, for instance, relate to an organization in which the machine-learned model is to be deployed. In particular, the local sites may relate to healthcare environments, organizations, or facilities such as hospitals, laboratories, practices, universities, or associations of one or more of the previously mentioned. According to some examples, the model aggregator device is located outside of the local sites and serves one or more of the local sites from the outside.

The machine-learned model may be conceived as a master model in a federated learning scheme which is centrally administrated by the model aggregator device.

In general, a machine-learned model is configured to provide a desired or predetermined kind of output by processing a certain kind of input data. Thereby, a machine-learned model mimics cognitive functions that humans associate with other human minds. In particular, by training based on training data, the machine-learned function is able to adapt to new circumstances and to detect and extrapolate patterns. Other expressions for machine-learned model may be trained function, trained machine learning model, trained mapping specification, mapping specification with trained parameters, function with trained parameters, algorithm based on artificial intelligence, or machine learned algorithm.

In general, parameters of a machine-learned model can be adapted by training so as to obtain a model update (e.g., in the form of a local update or a central update of the machine learned model at the model aggregator device). In particular, supervised training, semi-supervised training, unsupervised training, reinforcement learning and/or active learning can be used. Furthermore, representation learning can be used. In particular, the parameters of the machine-learned model can be adapted iteratively by several steps of training.

In particular, the machine-learned model can comprise a neural network, a support vector machine, a decision tree and/or a Bayesian network, and/or the trained function can be based on k-means clustering, Q-learning, genetic algorithms, a transformer network and/or association rules. In particular, a neural network can be a deep neural network, a convolutional neural network, or a convolutional deep neural network. Furthermore, a neural network can be an adversarial network, a deep adversarial network and/or a generative adversarial network. Moreover, a neural network can comprise a transformer network.

The local data may comprise (training) input data and, optionally, corresponding (training) output data. The training output data may be data the machine-learned function is expected to produce based on the input training data. The training output data may comprise verified outputs. According to some examples, the verified outputs may by verified by a (human) expert at the local site. Of note, for unsupervised learning, no output training data is required.

The local data or parts of the local data may be subject to a data protection obligation restricting the transmission of the training data to the outside of the local site. As such, according to some examples, the local data cannot be accessed from the outside of the local site. In particular, the local data cannot be accessed by the model aggregator device.

According to some examples, the local data may relate to medical data of one or more patients. For instance, the local data may comprise laboratory test results and/or pathological data stemming from pathological imaging and/or medical image data generated by one or more medical imaging facilities such as computed tomography devices, a magnetic resonance system, an angiography (or C-arm X-ray) system, a positron-emission tomography system or the like and any combination thereof. Further, the local data may comprise supplementary information relating to a patient, such as diagnosis reports, information about administered treatments, information about symptoms and treatment responses, health progression and the like. Such information can be provided by ways of an electronic medical record (EMR), for instance. The local data may be stored locally in one or more databases of the local sites. The databases may be part of hospital information systems (HIS), radiology information systems (RIS), clinical information systems (CIS), laboratory information systems (LIS) and/or cardiovascular information systems (CVIS), picture archiving and communicating systems (PACS) or the like. From these databases, the local data can be accessed locally for training machine learned models (and the later regular use of the machine learned models after deployment). The local data may be subject to data privacy regulations which may prohibit that the local data leaves the local sites. The local data may, in particular, comprise data sets with which a machine learned model can be trained, validated, and tested. According to some examples, the local data may comprise data sets based on which the local update has been validated and/or testes. In other words, the local data may not comprise training data based on which an actual further training of the machine learned model was performed. Data sets may comprise input data and associated output data which can be used to evaluate the performance of a machine learned model during supervised learning. The output data may be verified results corresponding to the input data. The output data may be generated and/or verified by a human based on the input data.

According to some examples, the local data comprises a plurality of individual data items. Thereby each data item may comprise a training input data item and, optionally, a corresponding training output data item. For instance, a training input data item may relate to an individual medical image data set of a patient and a training output data item may relate to a corresponding detection result. Thus, the local data may be conceived as a set comprising the plurality of data items.

According to some examples, the local update of the machine learned model may comprise the machine learned model wherein one or more parameters of the machine-learned model have been adapted (or changed or optimized), in particular, based on the local data. In particular, the one or more adapted parameters, may comprise one or more adapted weights and/or adapted hyperparameters of the machine-learned model. “Generated at the local site based on local data” may comprise that the local updated has been trained and/or validated and/or tested using the local data. Specifically, the local data may be split into training data, validation data and test data. For an actual training (in the sense of adapting the machine learned model to generate a local update) a backpropagation scheme may be used based on an appropriate cost function and using the training data. Based on the validation data, the best performing local update out of several local updates may be selected at the local site. The specificity and the sensitivity may then be determined at the local site based on the test data. According to some examples, specificity and sensitivity may be included in the log file.

According to some examples, the parameterization may be conceived as data minimization of the original local data. According to some examples, the parameterization may have a reduced information depth as compared to the local data and, in particular, a reduced data size. The parameterization may not be subject to the data protection obligation. The parameterization may comprise one or more parameters such as numerical values or semantic expressions characterizing the local data. According to some examples, the parameterization does not comprise the (original) local data and/or (only) excerpts of the local data. In particular, the parameterization may be configured to enable a reconstruction of at least parts of the local data and, in particular, those parts which are not subject to a data protection obligation.

According to some examples, the parameterization may be based on the training input data or the training input data and the training output data. According to some examples, the parameterization may be based on the training output data (only). This is because the expected output encoded in the local training output data may already reflect the contents of the training input data on a higher level and, thus, may provide a good basis for generating the synthetic representations. According to other examples, the parameterization may be based on training output data and corresponding excerpts of the training input data. To provide an example, the parameterization may comprise a description of a medical finding (which would be the training output data) and a snippet of a medical image data set showing the medical finding.

To provide an example from the field of medical imaging, the parameterization may not comprise the full image data of a medical image data set but merely certain key parameters. For instance, this may comprise the imaging modality and imaging parameters used, image quality metrics, locations and descriptions of findings, image snippets, and so forth. Noteworthy, it may not comprise any data based on which an identification of the patient may be possible. Trivially, this may be demographic information of the patient but also more subtle treats which could lead to an identification such as body shapes or implants visible in the medical image.

According to some examples, the synthetic representation may be conceived as a re-creation or re-construction of the original local data based on the parameterization. According to some examples, the synthetic representation is not subject to the data protection obligation. Still, according to some examples, the synthetic representation may comprise the relevant characteristics for evaluating and/or training the machine-learned model.

The generative AI function is a machine learned function or model which is configured to generate text, images, or other data based on input data. According to some examples, the input may be the parameterization, or a natural language prompt generated based on the parameterization. In other words, the generative AI function swaps the parameterization for the synthetic representation. According to some examples, the generative AI function may comprise a transformer network, in particular, a transformer-based (deep) neural network. According to some examples, the local data may comprise image data, the parameterization does not comprise the image data, and the generative AI function is a parameterization-to-image function outputting a synthetic version/representation of the image data. According to some examples, the generative AI function comprises a vision transformer as herein described.

The generative AI-function may be trained based on pairs of parameterizations and corresponding data, in particular, comprising image data. Thereby, the parameterizations may have been extracted from the corresponding data in the same or a similar fashion as the parameterization will be extracted from the local data. During training of the generative AI-function, the corresponding data may be used as a ground truth against which the output of the generative AI-function is compared.

According to some examples, the generative AI function may comprise a transformer network. A transformer network is a neural network architecture generally comprising an encoder, a decoder, or both an encoder and decoder. In some instances, the encoders and/or decoders are composed of several corresponding encoding layers and decoding layers, respectively. Within each encoding and decoding layer is an attention mechanism. The attention mechanism, sometimes called self-attention, relates data elements (such as words or pixels) within a series of data elements to other data elements within the series.

For a review on transformer networks, reference is made to Vaswani et al., “Attention Is All You Need”, in arXiv: 1706.03762, Jun. 12, 2017, the contents of which are herein included by reference in their entirety.

According to some examples, off-the-shelf generative AI functions may be used such as DALL-E or Midjourney. According to other examples, customized generative AI functions based on a transformer architecture may be used which are trained based on pairs of parameterizations and corresponding (real) data.

According to some examples, evaluating (another word is testing) may comprise testing if the local update is able to perform sufficiently well on unseen data. According to some examples, evaluating may comprise letting the machine-learned model process the synthetic representation and or other test data available at the model aggregator device and compare the outcome of the processing with the desired outcome. The other test data may comprise one or more synthetic representations obtained from parameterizations received from one or more local sites different than the local site.

The features synergistically work together to provide the model aggregator device with synthetic representations of the local data while safeguarding that the data protection requirements of the local sites are fulfilled. Further, the data minimization also reduces the amount of data which needs to be transferred which reduces the latency in the system. This allows for a more efficient federated learning workflow in distributed environments.

According to some examples, the step of updating the machine-learned model at the model aggregator device comprises aggregating the local update in the machine-learned model. With that a master model can successively optimized.

According to some examples, aggregating may comprise taking over one or more updated parameters of the local update in the machine-learned model. According to some examples, updating may be performed if the evaluation result indicates an increased performance of the local update.

According to some examples, the parameterization is generated by applying a trained feature encoder to the local data. According to some examples, the feature encoder is provided to the local site by the model aggregator device.

According to some examples, the parameterization comprises encoded features as identified by the feature encoder based on the local data. According to some examples, the encoder is different and/or independent from the generative AI function. This may mean that the encoder has been trained independently from the generative AI function and/or comprises a different architecture. According to some examples, generating the parameterization comprises applying the feature-encoder to each individual data item of the local data so as to generate, for each data item, a set of encoded features, and appending each set of encoded features to the parameterization/the log file.

According to some examples, the log file may comprise a performance log of the updated machine-learned function. The performance log may indicate how well the local update machine-learned model performed on the local data at the local site. According to some examples, the step of evaluating comprises additionally evaluating the local update based on the performance log. With that, an improved assessment of the local update can be made. As an alternative, the parameterization may be provided as such without including it in a log file. Then, no log file is generated/transmitted.

According to some examples, the log file is formatted according to the JSON standard. JSON stands for Javascript Object Notation and is an open standard file format and data interchange format that uses human-readable text to store and transmit data objects consisting of attribute-value pairs and arrays. With that, the interoperability of the method can be improved.

According to an aspect, the parameterization consists of a parameterization of the data used for validating and/or testing the local update at the local site.

In other words, only a parameterization of the data used for validating and/or testing the local update is generated/transmitted. The actual training data which was used for fine-tuning (in the sense of adapting) the machine learned model is not parameterized and/or no parameterization of such local data is transmitted according to this aspect. Thus, the parameterization does not comprise a parameterization of this actual training data. By consequence, the synthetic representation is (only) a synthetic recreation of the data sets used for testing and/or validating the local update but not of the training data sets.

This may have the advantage that the bias may be reduced as the model(s) are systematically not evaluated at the model aggregator device based on information they have been trained on.

According to an aspect, the generative AI function is configured to generate the synthetic representation based on a natural language prompt indicating the synthetic representation to be generated, and the step of generating comprises obtaining the natural language prompt based on the parameterization and inputting the natural language prompt in the generative AI function so as to generate the synthetic representation.

A prompt may be conceived as a natural language description of the synthetic representation to be generated.

Using prompt has the advantage that the method is readily compatible with off-the-shelf generative AI functions which often require prompts as input.

According to some examples, the step of generating comprises inputting the prompt together with the parameterization. This has the advantage that the generative AI function is provided with additional information for simulating the synthetic representation.

According to some examples, the prompts may be generated by a parser module configured to generate prompts based on parameterizations. The parser may comprise a language decoder configured to generate natural language text based on the parameterization. The language decoder may comprise a transformer network. By using a parser module, the workflow can be further automized and rendered more efficiently.

According to some examples, the method further comprises modifying the natural language prompt so as to generate a modified natural language prompt, wherein the step of generating the synthetic representation comprises inputting the modified prompt into the generative AI function so as generate a further synthetic representation and including the further synthetic representation in the synthetic representation.

In general, the modified prompt may comprise a different content and/or different instructions for the generative AI function.

With modified prompts, a further augmentation of the training data can be achieved. At the same time, the modified prompt may serve as ground truth for the further synthetic representation. For instance, an initial prompt may specify that an X-ray image displays a lesion at certain location in the lung of the patient. Then, the modified prompt may specify a lesion at a (slightly) different location.

According to some examples, the method further comprises adding the synthetic representation to an existing test data set for testing the machine learned model at the model aggregator device so as to generate an extended test data set, wherein, in the step of evaluating, the local update is evaluated based on the extended test data set.

In other words, the synthetic data is appended to the test data base of the model aggregator device. This may render the extended test data set more representative of the real-world scenarios and make it more resilient to concept drift in the local sites.

According to some examples, the method further comprises determining a data quality of the synthetic representation, wherein, in the step of evaluating, the local update is evaluated based on the data quality.

The data quality may comprise an indication how good the simulation of the local data at the model aggregator device (i.e., the synthetic representation) actually is. Specifically, the data quality may comprise an indication how well the synthetic representation corresponds to the local data.

According to some examples, the step of evaluating comprises checking if the data quality meets a predetermined quality criterion and using the synthetic representation for evaluating the local update if the data quality meets the predetermined quality criterion.

According to some examples, the step of adding the synthetic data comprises adding the synthetic representation based on the data quality. According to some examples, the step of adding the synthetic data comprises checking if the data quality meets a predetermined quality criterion and adding the synthetic data if it meets the predetermined quality criterion.

According to some examples, the parameterization is representative of a plurality of independent data items (of the local data), the step of generating a synthetic representation of the local data comprises generating a synthetic data item for each of the independent data items such that the synthetic representation comprises the synthetic data items.

According to some examples, the step of determining the data quality comprises determining a data quality measure for each synthetic data item.

According to some examples, the step of checking comprises checking if the data quality measure of each synthetic data item meets the predetermined criterion. According to some examples, the step of evaluating comprises using synthetic data items for evaluating the local update the data quality measure of which meets the predetermined criterion. According to some examples, the step of adding comprises adding synthetic data items to the existing test data the data quality measure of which meets the predetermined criterion.

By determining the data quality and using this information in the evaluation of the local update and/or the extension of the central test data set it can be ensured that inappropriate synthetic representations do not lead to unwanted effects.

According to an aspect, the step of determining a data quality of the synthetic representation comprises generating a backward parameterization of the synthetic representation, comparing the backward parameterization with the parameterization, and determining the data quality based on the step of comparing the backward parameterization with the parameterization.

According to some examples, the step of comparing comprises determining a difference or distance between the parameterization and the backward parameterization.

According to some examples, the backward parameterization is generated in substantially the same way as the parameterization. According to some examples the parameterization is generated at the local side by applying an encoder to the local data. According to some examples, the backward parameterization is generated, at the model aggregator device, by applying the encoder or a copy of the encoder to the synthetic representation.

According to some examples, the step of generating the backward parameterization comprises generating a backwards parameterization for each synthetic data item, and the step of comparing comprises comparing the backwards parameterizations with the respective parameterization (or the respective part of the parameterization) so as to generate a quality measure for each data item.

Due to the distributed environment a direct quality control of the synthetic representation is difficult. This is because the original training data typically has to stay at the local sites and is therefore not available for a comparison with the synthetic representation. Here, the suggested calculation of the backwards parameterization offers an elegant way to obtain a readout which can be directly compared to the information uploaded to the model aggregator device. With that, the quality of the synthetic representation can be ensured. In turn, this allows for a more efficient evaluation of machine learned functions in a federated learning setup.

According to an aspect, the step of determining a data quality of the synthetic representation comprises generating a natural language summary based on the synthetic representation, comparing the natural language summary to the natural language prompt, and determining the data quality based on the step of comparing the natural language summary to the natural language prompt.

According to some examples, the step of comparing comprises determining a difference or distance between the natural language summary and the natural language prompt.

According to some examples, the summary is generated by applying a parser to the synthetic representation which parser is configured to summarize and/or describe data in natural language text. The parser may comprise a language decoder configured to generate natural language text based on data, in particular, image data. The language decoder may comprise a transformer network. According to some examples, the parser may be of the same kind as or a copy of the parser used in generating the prompt.

According to an aspect, the step of generating the natural language summary comprises generating a natural language summary for each synthetic data item, and the step of comparing comprises comparing the natural language summaries to the respective natural language prompt so as to generate a quality measure for each data item.

By generating summaries based on the synthetic representations and comparing these summaries to the prompts which were used to trigger the creation of the synthetic representation another quality control of the method may be performed. The quality control based on the prompts may be carried out in addition to or as an alternative to the quality control based on the backwards parameterization.

According to some examples, the local data comprises a plurality of independent data items, and the parameterization comprises, for each independent data item, an item-parametrization of each data item, and one or more statistical properties of the plurality of independent data items.

According to some examples, the statistical properties may comprise one or more distributions of parameters of the independent data items and/or statistical observables derived from the distributions. A statistical observable may relate to quantifiable properties of a corresponding distribution. According to some examples, a statistical observable may comprise average values, the entropy, the skewness, the variance and so forth.

According to some examples, the one or more statistical properties are input into the generative AI function together with the respective item-wise parameterization(s) and the synthetic representation is generated additionally based on the one the one or more statistical properties. In particular, the synthetic representation may be generated such that the corresponding statistical properties of the synthetic representation correspond to the statistical properties of the parameterization.

Using the statistical properties may have the benefit that the synthetical representation more accurately corresponds to the local data. In particular, it can be ensured that the synthetic representation shows the same statistics as the local data.

According to some examples, the step of determining a data quality of the synthetic representation comprises determining one or more corresponding statistical properties based on the synthetic representation and comparing the corresponding statistical properties with the statistical properties. The corresponding statistical properties may comprise the same statistical observables as the statistical properties.

In other words, a quality control based on a statistical test may be implemented. This allows for an efficient way of supervising the quality of the synthetic representation in a scenario where the original training data is not available for a direct comparison.

According to an aspect, the method further comprises generating a modified parameterization based on the parameterization, wherein, in the step of generating, the synthetic representation is additionally generated based on the modified parameterization.

According to some examples, the modified parameterization may comprise one or more values which are different from the parameterization. According to some examples, the modified parameterization is configured in a way that it leads to (slightly) different synthetic representations if processed with the generative AI function as compared to the (original) parameterization. If, for instance, a section of the parameterization sets out a finding of certain characteristics, the corresponding section in the modified parameterization may set out a finding with different characteristics or no finding at all. Especially the latter is easy to implement, fail-safe and can boost the number of normal, i.e., unsuspicious samples, in the synthetic data.

According to some examples, the modified parameterization may be generated using a trained function which has been configured to derive (physically or medically) reasonable modifications for the parameterization. According to some examples, a corresponding trained function may be trained by relying on the quality control herein described until the trained function is capable of producing acceptable modified parameterizations.

In other words, a data augmentation of the parameterization is performed. In turn, this leads to additional and more varied data for testing the machine-learned model. Moreover, the perturbation may have the advantage that those data sets which were used for the actual adaptation of the machine learned model may be morel readily used in the evaluating step without introducing too much bias.

According to some examples, the data quality control measures described in connection with the synthetic representation generated based on the parameterization may also be applied to the synthetic representation generated based on the modified parameterization.

Specifically, the step of determining a data quality of the synthetic representation comprises determining one or more corresponding statistical properties based on the synthetic representation (generated based on the modified parameterizations) and comparing the corresponding statistical properties with the statistical properties of the (original) parameterization. With that, it can be checked if the modified parameterizations still lead to the same statistics.

According to some examples, the local data comprises a plurality of independent data items and the parameterization comprises, for each independent data item, an item-parametrization, wherein the step of generating a modified parameterization comprises generating modified item-parameterizations, and the step of generating the synthetic representation comprises generating a synthetic data item for each one of the item-parameterization and the modified item-parameterization wherein the synthetic representation comprises the synthetic data items.

According to some examples, the local data comprises protected information, in particular protected personal information, and the parameterization does not comprise the protected information.

According to some examples, the protected information may be information which must not leave the local site. For instance, the local data may be subject to a data protection regulation such as a confidentiality agreement or a legal regulation such as the General Data Protection Regulation of the EU.

Stripping the protected information for generating the parameterization allows to distribute such data to the outside of the local site. This enables using this information for testing the machine-learned model at the model aggregator device.

According to an aspect, the machine learned model is an image processing function configured to generate an image processing result based on image data, the local data comprises training image data, the parameterization comprises a parameterization of the training image data, the synthetic representation comprises synthetic image data generated by the generative AI function based on the parameterization of the training image data.

The local data comprises (training) image data and verified image processing results. For instance, the image data may comprise pictures acquired by a camera system or other image acquisition systems. According to some examples, this may relate to image data acquired by a smart phone camera, or a camera system mounted on a car. The image processing result may relate to an object detected in the image data such as persons, text, cars, lane markings, or other objects.

According to some examples, the parameterization comprises basic characteristics of the image data such as the resolution, color, noise levels, acquisition system and so forth. Further, it may comprise information characterizing the content of the underlying image data. According to some examples, this information may comprise feature vectors or embeddings extracted by an accordingly configured encoder. Further, according to some examples, the information may comprise one or more semantic meanings and relations of what is depicted in the image data. The parameterization may be tangible for a human (e.g., “the picture shows a person riding on a bike”) and/or (only) machine-interpretable information such as complex feature vectors. Moreover, the parameterization may comprise information about the verified image processing result (i.e., the training output data). According to some examples, the parameterization of the image data may be identical to the parameterization of the image processing result.

According to some examples, the parameterization may have been generated by applying an encoder or feature encoder to the local data. According to some examples, the feature encoder may comprise a vision transformer. The vision transformer may be configured to break down input images into patches and tokenize them (extracting representation vectors), before applying the tokens to a standard transformer architecture. The vision transformer may comprise an attention mechanism configured to repeatedly transform representation vectors of image patches for incorporating more and more semantic relations between image patches in an image.

According to some examples, the vision transformer and/or the generative AI function may be obtained by training a masked autoencoder. A masked auto-encoder comprises two vision transformers put end-to-end. The first one takes in image patches with positional encoding, and outputs vectors representing each patch. The second one takes in vectors with positional encoding and outputs image patches again. During training, both vision transformers are used. An image is cut into patches. The second vision transformer takes the encoded vectors and outputs a reconstruction of the full image. During use, the first vision transformer may be used as encoder and/or the second vision transformer may be used as generative AI function. With that, encoder and generative AI function complement one-another by design which enables a seamless data processing with limited losses.

According to some examples, the vision transformer and/or the generative AI function may be obtained or may be based on training a vision transformer VQGAN. In a vision transformer VQGAN there are two vision transformer encoders and a discriminator. One encodes patches of an image into a list of vectors, one for each patch. Another encodes the quantized vectors back to image patches. The training objective attempts to make the reconstruction image (the output image) faithful to the input image. The discriminator (usually a convolutional network, but other networks are possible) attempts to decide if an image is an original real image, or a reconstructed image by the vision transformer.

This has the advantage that, after such a vision transformer VQGAN is trained, it can be used to code an arbitrary image into a list of symbols, and code an arbitrary list of symbols into an image. The list of symbols can be used to train into a standard autoregressive transformer, for autoregressively generating an image. Further, one can take a list of caption-image pairs, convert the images into strings of symbols, and train a standard GPT-style transformer. Then at test time, one can just give an image caption, and have it autoregressively generate the image.

According to some examples, the synthetic image data is generated so that it resembles or mimics the local data as much as possible.

By parameterizing and reconstructing image data, an efficient federated learning scheme can be provided. Specifically, the method ensures data accessibility while safeguarding data privacy and reducing data traffic.

According to some examples, the parameterization does not comprise image data.

This may have the advantage of a particular efficient data minimization.

According to some examples, the parameterization may comprise one or more image patches extracted from the local data. In other words, the parameterization may comprise a subset of the image data in the local data. According to some examples, the patches may correspond to the image detection results. Specifically, a patch may be a cutout of the image data. In other words, the parameterization may only comprise the most relevant image data while less relevant parts of the image data are not included in the parameterization.

This may have the advantage that a more accurate synthetic representation of the image data can be made at the model aggregator device while still allowing for an appropriate data minimization.

According to some examples, the machine learned model is configured to generate an image processing result based on medical image data, the image processing result being selected from: a detection result of a medical finding in medical image data, a classification of a medical finding in medical image data, and/or a segmentation of medical image data, and the training image data comprises medical image data.

Accordingly, the synthetic representation may comprise a synthetic re-creation of the medical image data.

According to some examples, the medical image data comprises a plurality of medical image data sets respectively showing a body part of a patient.

The medical image dataset may relate to a medical image study. The medical image dataset may relate to three-dimensional data sets providing three dimensions in space or two dimensions in space and one dimension in time, to two-dimensional data sets providing two dimensions in space, and/or to four-dimensional data sets providing three dimensions in space and one dimension in time.

The medical image dataset may depict a body part of a patient in the sense that it contains three-dimensional image data of the patient's body part. The medical image dataset may be representative of an image volume. The patient's body part may be comprised in the image volume.

The medical image dataset comprises image data, for example, in the form of a two- or three-dimensional array of pixels or voxels. Such arrays of pixels or voxels may be representative of intensity, absorption, or other parameters as a function of three-dimensional position, and may, for example, be obtained by suitable processing of measurement signals obtained by a medical imaging modality.

A medical imaging modality corresponds to a system used to generate or produce medical image data. For example, a medical imaging modality may be a computed tomography system (CT system), a magnetic resonance system (MR system), an angiography (or C-arm X-ray) system, a positron-emission tomography system (PET system), an ultrasound imaging system or the like. Specifically, computed tomography is a widely used imaging method and makes use of “hard” X-rays produced and detected by a specially rotating instrument. The resulting attenuation data (also referred to as raw data) is presented by a computed analytic software producing detailed images of the internal structure of the patient's body parts. The produced sets of images are called CT-scans which may constitute multiple series of sequential images to present the internal anatomical structures in cross sections perpendicular to the axis of the human body. Magnetic Resonance Imaging (MRI), to provide another example, is an advanced medical imaging technique which makes use of the effect magnetic field impacts on movements of protons. In MRI machines, the detectors are antennas, and the signals are analyzed by a computer creating detailed images of the internal structures in any section of the human body.

Accordingly, the depicted body part of the patient in general will comprise a plurality of anatomies and/or organs (also denoted as compartments or anatomic structures). Taking a chest image as an example, the medical image dataset may show lung tissue, bones, e.g., the rib cage, heart and aorta, lymph nodes and others.

The medical image dataset may comprise a plurality of images or image slices. The slices may respectively show a cross-sectional view of the image volume. The slices may comprise a two-dimensional array of pixels or voxels as image data. The arrangement of slices in the medical image data set may be determined by the imaging modality or by any post-processing scheme used.

Further, the medical image dataset may be a two-dimensional pathology image data set, i.e., a so-called whole-slide image, depicting a tissue section of a patient.

According to some examples, the medical image data set may have been acquired at the local site.

The medical image data set may be stored in a standard image format such as the Digital Imaging and Communications in Medicine (DICOM) format and in a memory or computer storage system at the local site such as a Picture Archiving and Communication System (PACS). Whenever DICOM is mentioned herein, it shall be understood that this refers to the “Digital Imaging and Communications in Medicine” (DICOM) standard, for example according to the DICOM PS3.1 2020c standard (or any later or earlier version of said standard).

According to some examples the local data set comprises, for each medical image data set, a verified image processing result, in particular, a verified detection result of a medical finding in the respective medical image data set, a verified classification result of a medical finding in the respective medical image data set, and/or a segmentation of the respective medical image data set.

According to some examples, the parameterization comprises the verified image processing result or a parameterization thereof. As the case may be, the parameterization may comprise an indication of the medical imaging modality (or modalities) with which the medical image data has been acquired, imaging parameters used upon acquiring the medical image data, a medical finding comprised in the medical image data, a segmentation of an object comprised in the medical image data.

A medical finding may indicate a certain condition or pathology of the patient. The condition or pathology may be relevant for the diagnosis of the patient.

A medical finding may relate to an anatomical structure that differentiates the patient from other patients. Medical findings may be located within different organs of the patient (e.g., within the lung of a patient, or within the liver of a patient) or in between the organs of the patient. In particular, a medical finding may also relate to a foreign body.

In particular, a medical finding may relate to a neoplasm (also denoted as “tumor”), in particular, a benign neoplasm, an in-situ neoplasm, a malignant neoplasm and/or a neoplasm of uncertain/unknown behavior. In particular, a medical finding may relate to a nodule, in particular, a lung nodule. In particular, a medical finding may relate to a lesion, in particular, a lung lesion.

A classification may relate to identify a finding type and or to provide a classification according to a plurality of predefined classes such as benign or malignant.

According to some examples, a segmentation may be directed to an organ, finding, or other compartment of a body part of the patient. According to some examples, the step of segmenting may mean obtaining an outline of the respective organ or compartment and/or delineating the organ or compartment from the rest of the image data.

By applying the method to medical image data processing, the advantages of the method in particular take effect. This is because the medical environment is particularly regulated with restrictive data protection policies. At the same time there are tight regulations regarding the quality and validation of machine learned functions.

According to some examples, the parameterization does not comprise protected health information. Protected health information may in particular relate to personal information or other information of the patient which could lead to an identification of the patient.

According to some examples, the parameterization comprises one or more cutouts of the medical image data respectively made around a finding depicted in the medical image data. Another word for cutout may be patch. A cutout will generally display the finding and surrounding tissue but not the entire medical image. With that, better synthetic representations can be generated which more closely reflect the original local data.

According to an aspect, the method further comprises providing the updated machine learned model to a second local site different to the local site.

By providing the updated machine learned model to other sites, the knowledge gathered at one site through a local model update and centrally verified at the model aggregator device may be shared and distributed. At the second local site, a further local model update may be generated based on second local data. The further local update may be received at the model aggregator device together with a parameterization of the second local data and the process may start over for the further local update.

According to an aspect, a computer-implemented method for federated learning of a machine learned model is provided. The method comprises a plurality of steps. A first step is directed to receive, at a local site from a model aggregator device remote from the local site, a machine learned model. A further step is directed to generate (or provide), at the local site, a local update of the machine learned model using local data of the local site. A further step is directed to generate, at the local site, a parameterization of the local data. A further step is directed to transmit, by the local site, the local update and the parameterization to the model aggregator device.

According to a further aspect, a computer-implemented method for federated learning of a machine learned model is provided. The method comprises a plurality of steps. A first step is directed to receive, at a local site from a model aggregator device remote from the local site, a machine learned model (and, optionally, the generative AI function). A further step is directed to generate (or provide), at the local site, a local update of the machine learned model using local data of the local site. A further step is directed to generate, at the local site, a parameterization of the local data. A further step is directed to generate, at the local site, a synthetic representation of the local data based on the parameterization using a generative AI function. A further step is directed to transmit, by the local site, the local update and the synthetic representation to the model aggregator device.

In other words, the above method is directed to the client-side processing. The steps may be further detailed and combined with other features according to the aspects and examples herein described. In particular, the steps of the client-side processing may be combined with steps of the server-side processing at the model aggregator device. The advantages described in connection with the other aspects and examples of the disclosure are also realized by the correspondingly configured steps of the client-side processing.

According to an aspect, a computer-implemented method for providing a synthetic representation of local data at a local site to an aggregator device is provided. The method comprises a plurality of steps. One step is directed to generate at the local site a parameterization of the local data. Another step is directed to transmit the parameterization from the local site to the aggregator device. Another step is directed to receive the parameterization at the aggregator device. Another step is directed to generate, at the aggregator device, a synthetic representation of the local data based on the parameterization using a generative AI function. Another step is directed to provide the synthetic representation at the aggregator device.

According to an alternative aspect, a computer-implemented method for providing a synthetic representation of local data at a local site to an aggregator device is provided, the method comprising generating, at the local site, a synthetic representation of the local data using a generative AI function and providing the synthetic representation from the local site to the aggregator device. Specifically, generating the synthetic representations may at the local sites may comprise generating, at the local site, a parameterization of the local data and generating, at the local site, a synthetic representation of the local data based on the parameterization using the generative AI function.

With the above methods, a data privacy preserving way of exchanging information may be provided. The aggregator device may be configured in an equivalent way as the model aggregator device. Further the above method may be modified according to the other examples and aspects herein described and may entail similar advantages.

According to an aspect, a model aggregator device for federated learning of a machine learned model is provided, which model aggregator device comprises a computing unit and an interface unit. The interface unit is configured to receive, from a local site remote from the model aggregator device, a local update of the machine learned model and a log file, wherein the local update was generated at the local site based on local data and the log file comprises a parameterization of the local data. The computing unit is configured to generate a synthetic representation of the local data based on the parameterization using a generative AI function, to evaluate the local update using the synthetic representation so as to obtain an evaluation result indicative of the performance of the model update, and to update the machine learned model based on the evaluation result and local update.

The computing unit may be realized as a data processing system or as a part of a data processing system. Such a data processing system can, for example, comprise a cloud-computing system, a computer network, a computer, a tablet computer, a smartphone and/or the like. The computing unit can comprise hardware and/or software. The hardware can comprise, for example, one or more processors, one or more memories, and combinations thereof. The one or more memories may store instructions for carrying out the method steps according to embodiments of the present invention. The hardware can be configurable by the software and/or be operable by the software. Generally, all units, sub-units or modules may at least temporarily be in data exchange with each other, e.g., via a network connection or respective interfaces. Consequently, individual units may be located apart from each other. Further, the computing unit may be configured as an edge device.

The interface unit may comprise an interface for data exchange with one or more local clients, e.g., via the Internet. The interface unit may be further adapted to interface with one or more users of the system, e.g., by displaying the result of the processing to the user (e.g., in a graphical user interface).

The model aggregator device may be adapted to implement the methods as herein described in their various aspects and examples for the federated learning of a machine learned function. The advantages described in connection with the method aspects and examples may also be realized by the correspondingly configured systems' components.

According to an aspect, a local model update device for federated learning of a machine learned model is provided. The local model update device is located at a local site. The local model update device comprises a local interface unit and a local computing unit. The local interface unit is configured to receive, from a model aggregator device remote from the local site, a machine learned model, and to transmit, a local update of the machine learned model and a parameterization of the local data used for generating the local update at the local site to the model aggregator device. The computing unit is configured to generate, at the local site, the local update of the machine learned model based on the local data, and to generate the parameterization of the local data.

One or more of the local model update devices may be combined with the model aggregator device to form a system for federated learning of the machine learned function. The local computing unit(s) may generally be configured in an equivalent manner as the computing unit. Likewise, the local interface unit(s) may be configured in the substantially the same way as the interface unit.

According to another aspect, the present invention is directed to a computer program product comprising program elements which induce a computing unit of a model aggregator device (or a local model update device) for federated learning of a machine learned function to perform the steps according to one or more of the above method aspects and examples, when the program elements are loaded into a memory of the computing unit.

According to another aspect, the present invention is directed to a computer-readable medium on which program elements are stored that are readable and executable by a computing unit of a model aggregator device (or a local model update device) for federated learning of a machine learned function to perform the steps according to one or more method aspects and examples, when the program elements are executed by the computing unit.

The realization of embodiments of the present invention by a non-transitory computer program product and/or a non-transitory computer-readable medium has the advantage that already existing providing systems can be easily adapted by software updates in order to work as proposed by embodiments of the present invention.

The computer program product can be, for example, a computer program or comprise another element next to the computer program as such. This other element can be hardware, e.g., a memory device, on which the computer program is stored, a hardware key for using the computer program and the like, and/or software, e.g., a documentation or a software key for using the computer program. The computer program product may further comprise development material, a runtime system and/or databases or libraries. The computer program product may be distributed among several computer instances.

### BRIEF DESCRIPTION OF THE DRAWINGS

Characteristics, features and advantages of the above-described invention, as well as the manner they are achieved, become clearer and more understandable in the light of the following description of embodiments, which will be described in detail with respect to the figures. This following description does not limit the present invention on the contained embodiments. Same components, parts or steps can be labeled with the same reference signs in different figures. In general, the figures are not drawn to scale. In the following:

FIG. 1 schematically depicts an embodiment of a system for federated learning of a machine learned function according to an embodiment;

FIG. 2 schematically depicts a method for federated learning of a machine learned function according to an embodiment;

FIG. 3 schematically depicts an exemplary data flow diagram in connection with a method for federated learning of a machine learned function according to an embodiment;

FIG. 4 schematically depicts an exemplary data flow diagram in connection with a method for federated learning of a machine learned function according to an embodiment;

FIG. 5 schematically depicts a method for federated learning of a machine learned function according to an embodiment;

FIG. 6 schematically depicts an exemplary data flow diagram in connection with a method for federated learning of a machine learned function according to an embodiment; and

FIG. 7 schematically depicts an encoder-decoder transformer network according to an embodiment.

### DETAILED DESCRIPTION

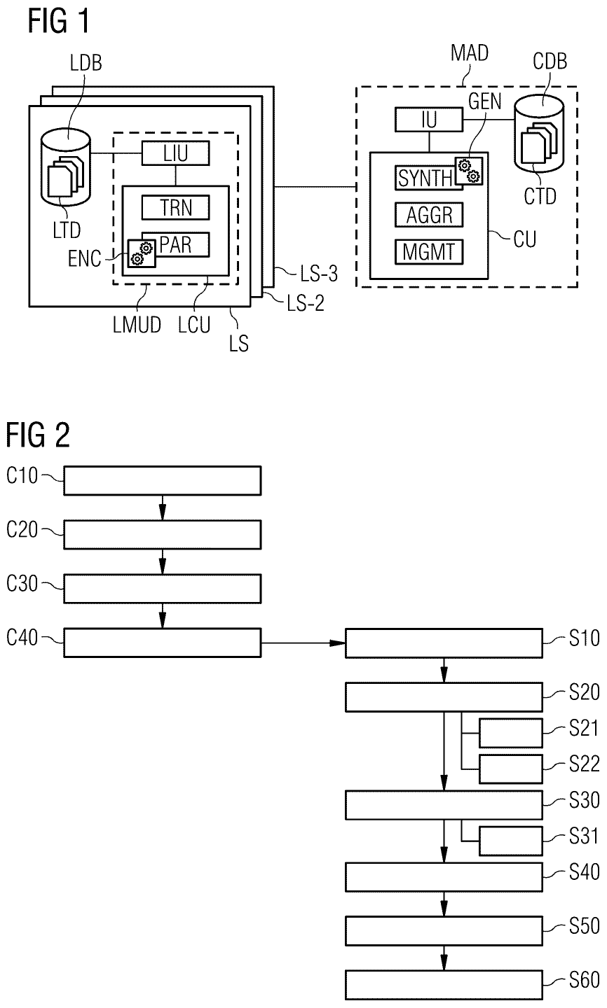

FIG. 1 depicts an example system 1 for federated learning of a machine learned model ML in a distributed environment. The system may be capable of creating, training, updating, dis-tributing, monitoring and, generally, administrating the machine-learned model ML in an environment comprising a plurality of local sites LS, LS-2, LS-3. System 1 is adapted to perform the method according to one or more embodiments, e.g., as further described with reference to FIGS. 2 to 6.

System 1 comprises a model aggregator device MAD and a plurality of clients respectively located at different local sites LS, LS-2, LS-3. The model aggregator device MAD and the clients may be interfaced via a network. The model aggregator device MAD is generally configured to control, coordinate, and steer the federated learning procedures in system 1. The local sites LS, LS-2, LS-3 may, for instance, relate to clinical or medical environments, such as hospitals or hospital groups, clinics or practices.

The machine learned model ML can be conceived as a master model which is centrally administrated by the model aggregator device MAD and distributed to and further trained at the local sites LS, LS-2, LS-3. The machine learned model ML may generally be configured provide a medical diagnosis based on medical input data. This may include outcome prediction, the detection of findings in medical image data, the annotation of medical images, e.g., in terms of orientations or landmark detection, the generation of medical reports and the like.

The model aggregator device MAD may be hosted on a server, which may be a cloud server or a local server. However, the model aggregator device MAD also may be implemented using any other suitable computing device(s). The model aggregator device MAD comprises a computing unit CU and an interface unit IU. Further, the model aggregator device MAD may have access to a central database CDB which is configured for centrally storing training data for evaluating the machine learned model ML.

The computing unit CU may comprise one or more processors and a working storage. The one or more processor(s) may include, for example, one or more central processing units (CPUs), graphics processing units (GPUS), and/or other processing de-vices. Computing unit CU may further comprise a micro-controller or an integrated circuit. Alternatively, computing unit CU may comprise a real or virtual group of computers like a so called ‘cluster’ or ‘cloud’. The working storage may include one or more computer-readable media such as a RAM for temporally loading data, e.g., data from the database CDB or data uploaded from the local sites LS, LS-2, LS-3. The working storage may further store information accessible by the one or more processors, for performing method steps according to one or more embodiments herein described.

The interface unit IU can include any suitable components for interfacing with one or more networks, including, for example, transmitters, receivers, ports, controllers, or other suitable components. The model aggregator device MAD may exchange information with one or more local sites LS, LS-2, LS-3 via the interface unit IU. Any number of local sites LS, LS-2, LS-3 can be connected to model aggregator device MAD over the interface unit IU.

Computing unit CU may comprise sub-units SYNTH, AGGR, and MGMT. Sub-unit MGMT may be a management module or unit configured for controlling and administrating the federated learning of the machine learned model ML in the system 1. Sub-unit MGMT may trigger the distribution of the machine-learned model ML to the local sites LS, LS-2, LS-3 and initiate the update of machine learned model ML in the system 1 once a new updated version ML* is available.

Sub-unit SYNTH may be conceived as a training data synthesizer. Sub-unit SYNTH is configured to generate synthetic representations SR of the local data LTD based on corresponding parameterizations P. To this end, sub-unit SYNTH may be configured to host and run an accordingly configured generative AI function GEN.

Sub-unit AGGR may be seen as a model update unit. Sub-unit AGGR is configured to evaluate local model updates ML′ and aggregate the local updates ML′ in the master machine learned model ML if they constitute an improvement. To this end, sub-unit AGGR may be configured to apply a cross-validation scheme.

The designation of the distinct sub-units SYNTH, AGGR, MGMT is to be construed by ways of example and not as limiting the disclosure. Accordingly, sub-units SYNTH, AGGR, MGMT may be integrated to form one single processing unit or can be embodied by computer code segments configured to execute the corresponding method steps running on a processor or the like of computing unit CU. Each sub-unit SYNTH, AGGR, MGMT may be individually connected to other sub-units and or other components of the system 1 where data exchange is needed to perform the method steps.

The central database CDB may be realized as a cloud storage. Alternatively, the central database CDB may be realized as a local or spread storage, in particular, within the premises of the model aggregator device MAD. The central database CDB is configured to store central training data CTD.

Each of the local sites LS, LS-2, LS-3 comprises a local model update device LMUD and a local database LDB. The local database LDB may be realized as a local or spread storage within the premises of the respective local site LS, LS-2, LS-3. The local database LDB may store the local (training) data LTD which is to be processed by the machine learned model ML.

The local data LTD may comprise a number of individual data items relating, for instance, to a clinical or medical problem. As an example, the data items may relate to laboratory test results and/or pathological data and/or medical imaging data, electronic medical records, and any combination thereof. The local data LTD may relate to medical data of one or more patients. The local data LTD may have been generated at the respective local sites LS, LS-2, LS-3. The local database LDB may be part of a hospital information systems (HIS), radiology information systems (RIS), clinical information systems (CIS), laboratory information systems (LIS) and/or cardiovascular information systems (CVIS), picture archiving and communicating system (PACS) or the like.

From the local database LDB, the local data LTD can be accessed locally for training the machine learned model ML and the later regular use of the machine learned model ML after deployment. Training may comprise adapting the machine learned model and validating and testing the adapted machine learned model. The local data may be split into training data, validation data and test data. For training the machine learned model at the local sites, the backpropagation algorithm may be used based on an appropriate cost function and using the training data. Based on the validation data, the best performing machine learned model out of several machine learned models (with different hyperparameters, e.g., number of layers, size, and number of kernels, padding etc.) may be selected. The specificity and the sensitivity may then be determined based on the test data.

In particular, the local data LTD cannot be accessed from the outside as the local data LTD may be subject to data protection regulations which may prohibit that the local data LTD leaves the local sites LS, LS-2, LS-3. The local data LTD may comprise training input data and associated training output data which can be used to evaluate the performance of a machine learned model ML during training. The output training data may relate to verified results corresponding to the input training data. The output training data may be generated and/or verified by a human based on the input training data.

The local model update device LMUD may comprise a local computing unit LCU and a local interface unit LIU. The local interface unit LIU may be configured in an equivalent way as the interface unit IU and may include any suitable components for interfacing with the interface unit IU over a network such as the internet.

The local computing unit LCU is configured to further train the machine learned model ML based on the local data LTD so as to provide a local update ML′ of the machine learned model ML. To this end, the local computing unit LCU may comprise an accordingly configured training unit or module TRN. Further, the local computing unit LCU may comprise a parameterization module or unit PAR which is configured to generate a parameterization P of the local data LTD. To this end, the parameterization unit PAR may be configured to host a correspondingly configured encoder function ENC. To ensure that no privacy sensitive information can be derived or inferred from the parameterization P, one or more encryption techniques, random noise techniques, and/or other security techniques can be added by the parameterization unit PAR upon generating parameterizations P. Both, the local update ML′ and the parameterization P may be provided to the model aggregator device MAD via the local interface unit LIU.

The designation of the distinct sub-units TRN, PAR is to be construed by ways of example and not as limiting the disclosure. Accordingly, sub-units TRN, PAR may be integrated to form one single processing unit or can be embodied by computer code segments configured to execute the corresponding method steps running on a processor or the like of the local computing unit LCU.

Local computing units LCU can be any suitable type of computing device, such as a general-purpose computer, special purpose computer, laptop, local server system, or other suitable computing device. The local computing units LCU may include one or more processor(s) and a memory. The one or more processor(s) may include, for example, one or more central processing units (CPUs), graphics processing units (GPUs), and/or other processing devices. The memory can include one or more computer-readable media and can store information accessible by the one or more processors, including instructions that can be executed by the one or more processors. The instructions can include instructions for the local further training of the machine learned model ML and/or the generation of parameterizations P.

In an alternative embodiment (not shown), it would also be conceivable to provide the local sites LS, LS-2, LS-3 with modules for generating synthetic representations SR of the local data LTD based on corresponding parameterizations P. Then, the synthetic representation SR would be generated directly at the local sites LS, LS-2, LS-3 and transmitted to the model aggregator device MAD. Also according to this alternative, data protection requirements may be complied with as only the synthetic representation SR leaves the local sites LS, LS-2, LS-3.

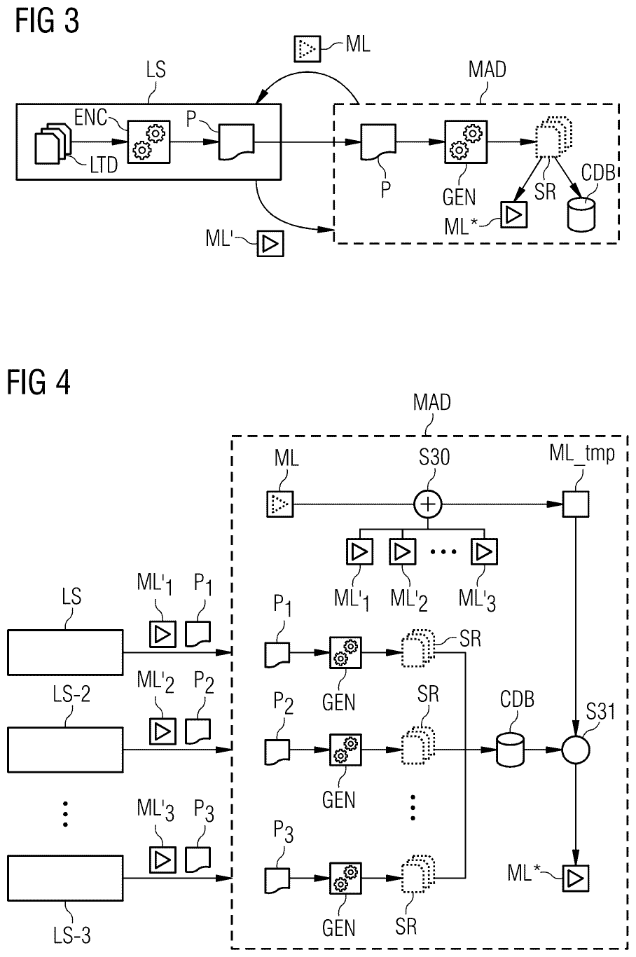

FIG. 2 depicts a method for federated learning of a machine learned model in a distributed environment. Corresponding data streams are illustrated in FIG. 3. Additionally,

FIG. 4 shows data streams associated with the model aggregation at the model aggregation device MAD. The method comprises several steps. The order of the steps does not necessarily correspond to the numbering of the steps but may also vary between different embodiments of the present invention. Further, individual steps or a sequence of steps may be repeated.

Steps C10-C40 happen at the client-side, i.e., at the respective local sites LS, LS-2, LS-3, and may be executed by the local model update device LMUD. Steps S10-S80 happen at the server-side and may be executed by the model aggregator device MAD. According to methods of the present inventions, the steps may be executed separately. In other words, aspects of embodiments of the present invention cover methods only comprising client-side methods steps, while other aspects cover methods only comprising server-side step. Further, aspects of embodiments of the present invention may also cover methods comprising server-side and client-side steps.

At step C10, a machine learned model ML is received at the local side LS. The machine learned model ML may be a copy of the master model provided and administrated by the model aggregator device MAD. The machine learned model ML is readily trained and meant to be deployed at the local site LS according to the learned task. According to embodiments, this learnt task may comprise an automated processing of medical image data for deriving a medical diagnosis. Specifically, the machine learned model may be configured to process medical image data of a patient in order to detect and/or classify medical findings. According to some examples, the medical image data may show parts of the patient's torso and the findings comprise lesions in the lung or liver of the patient. According to other examples, the medical image data comprise digital pathology images of the patient and the findings relate to a segmentation of the digital pathology image according to one or more tissue types.

At step C20, the machine learned model ML may be further trained based on the local data LTD at the local sites LS. This leads to a local update ML′ of the machine learned model ML. According to some examples, such training may happen on the fly, e.g., when a user at the local site reviews the processing results of the machine learned model ML. For instance, a radiologist may decline or accept lesions found by the machine learned model ML. Further, the radiologist may add lesions not found by the machine learned model. According to other examples, a pathologist may modify a segmentation as provided by the machine learned model ML. The user inputs may be used as ground truth for further optimizing, that is, training the machine learned model ML at the local sites LS, LS-2, LS-3. The ground truth together with the underlying local data may form the local data LTD.

At step C30, a parameterization P of the local data LTD or part of the local data may be generated. In particular, only the part of the local data used for validating the further trained machine learned model may be parameterized. The parameterization P may relate to a data minimization step in which the local data LTD is stripped down to a version which still allows to synthesize or re-create the local data LTD at the model aggregator device MAD, but which does not contain any unnecessary information. In particular, the parameterization P may not comprise any information which is subject to a data protection regulation such as personal information of a patient.

The parameterization P may comprise a plurality of characteristic values of the underlying medical image and, optionally, also image data excerpts/cutouts of the medical image. To provide an examples, the parameterization P may read as follows: type: Chest-CT scan, bolus agent: xyz, modality: Siemens Healthineers CT scanner, model number: 12345, kilovoltage peak: xxx, milliampere seconds: yyy, lung nodule 1: size: 11 mm, type: solid, location: upper left lung lobe, lung nodule 2: size: 16 mm, type: ggn, location: lower left lung lobe, etc. According to other examples, the parameterization P may have a more abstract form and may be provided in the form of embeddings which may be interpreted by the generative AI function GEN but not necessarily by a human user. Further, the parameterization P may also comprise one or more statistical properties of the entire local data.

At step C30, the parameterization P may be generated by an encoder ENC or auto-encoder which may be provided to the local site LS by the model aggregator device MAD together with the machine learned model ML. The encoder ENC may be seen as a counterpart of the generative AI function GEN and may be trained in conjunction with the generative AI function GEN as herein described.

At step C40, the local update ML′ and the parameterization P are transmitted to the model aggregator device MAD by the local model update device LMUD.

At step S10, the local update ML′ and the parameterization P are, in turn, received at the model aggregator device MAD. At step S20, a synthetic representation SR of the local data LTD is generated. To this end, generative AI function GEN may be applied to the parameterization P. According to the above example the synthetic representation SR, again, comprises a medical image such as a radiological or a pathological medical image as a recreation of the corresponding image data at the local site LS.

Optionally, the generative AI function GEN may function on the basis of a natural language prompt. The prompt may be seen as an instruction or control command for the generative AI function. Such prompt may be generated in optional sub-step S21 on the basis of the parameterization P. In a way step S21 may be seen as a translation step of translating the parameterization P into a set of instructions on the basis of which the generative AI function GEN may act.

At optional step S22, the prompt is input into the generative AI function so as to trigger the generation of the synthetic representation SR.

At step S30, the synthetic representation SR is used, optionally, together with other training already existing in the central data base CDB to evaluate the local update ML′. The result of the processing may be provided in the form of an evaluation result.

A data flow diagram of a model evaluation and aggregation process is shown in FIG. 4. As can be seen in FIG. 4, the model aggregator device MAD not only receives local updates ML′ from one local site LS but from a plurality of local sites LS, LS-1, LS-2. Likewise, the model aggregator device MAD may receive synthetic representations SR from different local sites LS, LS-1, LS-2.

For testing, validating and finally arriving at an updated master model ML*, a validation scheme may be used at sub-step S31. In particular, a cross-validation scheme may be used according to which the available data, that is, the synthetic representation SR and other appropriate training data available at the model aggregator device MAD, is partitioned into a plurality of complementary subsets or folds. Different available models (or their parameters) may be combined in a trail-fashion to generate a plurality of candidate model updates ML_tmp. Additional further training of these different combinations ML_tmp may be performed on one subset of the available data (called the training set or training fold) and testing is performed on the other subset (called the testing set or fold). To reduce variability, multiple rounds of cross-validation may be performed using different partitions of the training data, and the validation results are combined (e.g., averaged) over the different partitions to give an estimate of the respective machine learned model's ML_tmp predictive performance. If additional hyperparameters need to be optimized, nested cross validation schemes may be applied. Basically, these rely on (1) an inner cross-validation to tune the hyperparameters and select optimal hyperparameters, and (2) an outer cross validation used to evaluate the model trained using optimized hyperparameters as selected by the inner cross-validation. The best model may be provided in the form of a (final) evaluation result.

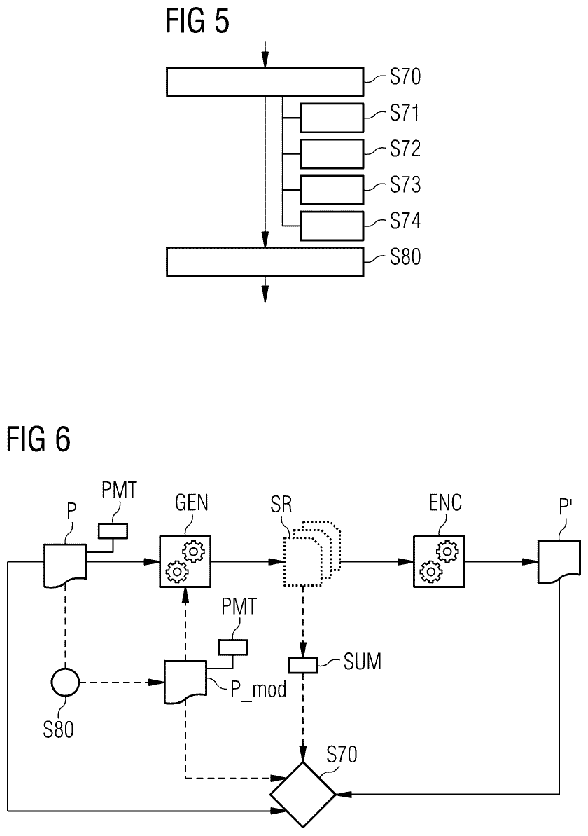

Before using synthetic representations SR in the evaluation of the machine learning model ML_tmp, they may be subjected to quality control as described in connection with FIGS. 5 and 6.

On the basis of the model evaluation and aggregation result of step S30, the best-performing variant may be adopted for the updated version of the master model and provided as the (global) update ML* of the machine learned model ML.

As can be seen from FIGS. 3 and 4, the method may further comprise a step of adding the synthetic representation SR to the central database CDB. This may happen at optional step S50. Further, the addition of synthetic representations SR to the central database CDB may be preceded by quality control steps for determining if the synthetic representation SR has sufficient quality. In this regard, the steps explained in connection with FIGS. 5 and 6 may be employed.

Finally, at optional step S60, the updated version ML* of the machine learned model ML may be rolled out to one or more of the local sites LS, LS-1, LS-2 so as to provide the (globally) updated machine learned model ML*.

FIG. 5 depicts optional sub steps in a method for federated learning of a machine learned model in a distributed environment. Corresponding data streams are illustrated in FIG. 6. The method comprises several steps. The order of the steps does not necessarily correspond to the numbering of the steps but may also vary between different embodiments of the present invention. Further, individual steps or a sequence of steps may be repeated.

At step S70 a quality assessment of the synthetic representation SR is performed. In the workflow depicted in FIG. 2, step S70 may, e.g., follow step S20. In step S70 two alternative processes for quality control are comprised. One involves the generation of a backwards parameterization P′ (steps S71 and S72), the other is based on the generation of a text summary SUM (steps S73 and S74). The two processes may respectively be applied separately or in combination.

Specifically, at step S71, a backward parameterization P′ of the synthetic representation SR may be generated. According to some examples, the same encoder ENC which was used to generate the parameterization P at the local site LS may be used.

That followed, at step S72, the backward parameterization P′ may be compared with the parameterization P. If, based on the comparison, the backward parameterization P′ and the parameterization P sufficiently correspond, it can be assumed that the data quality of the synthetic representation SR is sufficient for the further usage (e.g., an integration in the central database CDB and/or the evaluation/aggregation of the local update ML′).

At sub step S73, a textual summary SUM of the synthetic representation SR may be generated. For instance, the textual summary may be automatically generated by applying yet another trained function which is independent of the generative AI function. In the medical context, visions transformers may be used which have been trained to analyze medical image data and consolidate the findings into textual impressions (like the ones to be found in medical reports).

At step S74, the summary SUM may be compared to the prompt. If the textual summary SUM matches the prompt this may be seen as an indication for sufficient data quality of the synthetic representation SR.

Step S80 may be seen as an optional data augmentation step. In step S80, further variants of the parameterization P may be generated by tweaking or perturbing the values comprised. For instance, the size and location of nodules may be slightly varied. Further, descriptions of further nodules may be added while the description of other nodules may be deleted. In other words, this leads to perturbed parameterizations P mod for the generative AI function. As the case may be, the perturbed features may be used to generate additional prompts which deviate from the original prompt based on the unperturbed parameterization P.

According to other examples, the prompt PMT may also be perturbed/modified directly (if a prompt PMT is generated in workflow). Also in this case, further versions for the input in the generative AI function GEN may be obtained.

In turn, this leads to additional synthetic representations SR which may further increase the amount of data in the central database CDB. Self-speaking, the synthetic representations SR generated based on suchlike perturbed input parameters may be subjected to the same quality controls as described in connection with step S70 and shown in FIG. 6.

According to some examples, the encoder ENC used to generate the parameterization P may relate to an encoder part ENCP of an encoder-decoder transformer network and the generative AI function GEN may relate to the decoder part DEC of an encoder-decoder transformer network. The encoder part may be configured to take in images and output feature encodings while the decoder is configured to generate a synthetic representation of the input images based on the feature encodings. In other words, encoder ENC and generative AI function GEN may be seen as vision transformers which operate in a reciprocal manner with respect to one another. In this regard, the feature encodings which connect the two parts may be seen as the parameterization P of the present invention.

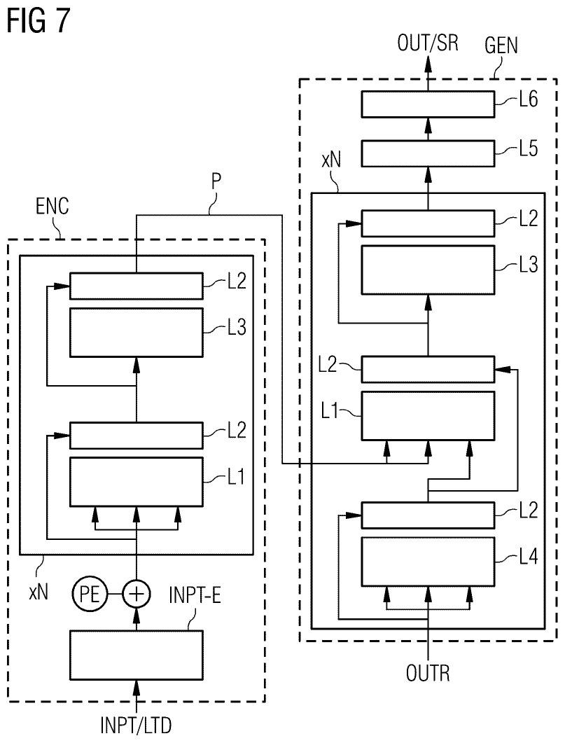

That said, FIG. 7 shows a schematic representation of an encoder-decoder transformer network according to an embodiment. While the usage of suchlike structure may have some advantages, like end-to-end training, it should be noted that other configurations may also be possible. In particular, the encoder ENC and the generative AI function GEN may also be independent from one another as elsewhere herein described.

In brief, the task of the encoder ENC is to map an input INPT, i.e., the local data LTD, in particular, a medical image, to a sequence of continuous representations, the parameterizations P, which are then fed into a decoder GEN. The decoder GEN receives the output P of the encoder ENC together with the decoder output OUTR at a previous iteration to generate an output OUT, which is a synthetized representation SR of the input INPT, in particular, a synthetic image.

The encoder ENC of this embodiment may comprise of a stack of N=8 identical layers. For the sake of easy reference, only one layer xN is shown in the drawing. Further, N may also be set to different values and, in particular, to values greater than N=8 according to the respective task. Each layer xN of the encoder ENCP comprises two sublayers L1 and L3. The first sublayer L1 implements a so-called multi-head self-attention mechanism. Specifically, the first sublayer L1 may be configured to determine how relevant a particular image data element is with regard to other elements in the input INPT. This may be represented as an attention vector. To avoid any bias, multiple attention vectors per word may be generated and fed into a weighted average to compute the final attention vector of every word. The second sublayer L3 is a fully connected feed-forward network which may, for example, comprise two linear transformations with Rectified Linear Unit (ReLU) activation in between. The N=8 layers of the encoder ENC apply the same linear transformations to all the elements in the input INPT, but each layer employs different weight and bias parameters to do so. Each sublayer L1, L3 is succeeded by a normalization layer L2, which normalizes the sum computed between the input fed into the respective sublayer L1, L3, and the output generated by the respective sublayer L1, L3 itself. In order to capture information about the relative positions of the elements in the input INPT, positional encodings PE are generated based on input embeddings INPT-E prior to being fed into the layers xN. The positional encodings PE are of the same dimension as the input embeddings INPT-E and may be generated using sine and cosine functions of different frequencies. Then, the positional encodings PE may be simply summed to the input embeddings INPT-E in order to inject the positional information PE. Input embeddings INPT-E may be, as usual, a representation of each image patch in the input INPT, typically in the form of a real-valued vector that encodes the pattern or other visual characteristics such that patches that are closer in the vector space are expected to be similar in. According to some examples, a convolutional neural network may be used to generate the input embeddings INPT-E.

The decoder GEN of this embodiment may also comprise of a stack of N=8 identical layers xN each comprising three sublayers L4, L1, L3 which may be succeeded by a normalization layer L2 as explained in connection with the encoder ENC. For the sake of easy reference, only one layer xN of the decoder GEN is shown in the drawing. Further, N may also be set differently and, in particular, greater than N=8 according to the respective task. While the sublayers L1 and L3 of the decoder GEN correspond in their functionality to the respective sublayers L1 and L3 of the encoder ENC, sublayer L4 receives the previous output OUTR of the decoder GEN (optionally transformed into corresponding embeddings and augmented with positional information if the output is a synthetized image patch), and implements multi-head self-attention over it weighing how important individual elements of the previous output vector OUTR are. That followed, the values from the first sublayer L4 of the decoder DEC are input in the L1-sublayer of the decoder GEN. This sublayer L1 of the de-coder GEN implements a multi-head self-attention mechanism similar to the one implemented in the first sublayer L1 of the encoder ENC. On the decoder side, this multi-head mechanism receives the values from the previous decoder sublayer L4 and the output of the encoder ENC. This allows the decoder GEN to attend to all the patches in parallel. Like in encoder ENC part, the output of the L1 sublayers is passed into a feed-forward layer L2, which will make the output vectors form into something which is easily acceptable by another decoder block or a linear layer. After all layers xN of the decoder DEC have been processed, the intermediate result is fed into a linear layer L5 which may be another feed-forward layer. It is used to expand the dimensions into an image format expected for the output OUT. That followed, the result is passed through a Softmax Layer L6, which transforms the result into a final output.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Wherever meaningful, individual embodiments or their individual aspects and features can be combined or exchanged with one another without limiting or widening the scope of the present invention. Advantages which are described with respect to one embodiment of the present invention are, wherever applicable, also advantageous to other embodiments of the present invention. Independent of the grammatical term usage, individuals with male, female or other gender identities are included within the term.

What is claimed is:

1. A computer-implemented method for federated learning of a machine learned model in a model aggregator device, the method comprising:
receiving, at the model aggregator device, from a first local site that is remote from the model aggregator device, a local update of the machine learned model and a parameterization of local data, wherein the local update was generated at the first local site based on the local data;
generating, at the model aggregator device, a synthetic representation of the local data based on the parameterization using a generative AI function;
evaluating, at the model aggregator device, the local update using the synthetic representation to obtain an evaluation result indicative of performance of the local update; and
updating, at the model aggregator device, the machine learned model based on the evaluation result and the local update.

2. The method according to claim 1, wherein
the parameterization includes a parameterization of data used for at least one of validating or testing the local update at the first local site.

3. The method according to claim 1, wherein
the generative AI function is configured to generate the synthetic representation based on a natural language prompt indicating the synthetic representation to be generated, and
the generating includes
obtaining the natural language prompt based on the parameterization, and
inputting the natural language prompt into the generative AI function to generate the synthetic representation.

4. The method according to claim 1, further comprising:
adding the synthetic representation to an existing test data set accessible for the model aggregator device for at least one of validating or testing the machine learned model to generate an extended test data set, and wherein
the evaluating evaluates the local update based on the extended test data set.

5. The method according to any claim 1, further comprising:
determining a data quality of the synthetic representation, and wherein
the evaluating evaluates the local update based on the data quality.

6. The method according to claim 5, wherein the determining a data quality of the synthetic representation comprises:
generating a backward parameterization of the synthetic representation;
comparing the backward parameterization with the parameterization; and
determining the data quality based on the comparing the backward parameterization with the parameterization.

7. The method according to claim 5, wherein the determining a data quality of the synthetic representation comprises:
generating a natural language summary based on the synthetic representation;
comparing the natural language summary to a natural language prompt indicative of the synthetic representation; and
determining the data quality based on the comparing the natural language summary to the natural language prompt.

8. The method according to claim 1, wherein
the local data includes a plurality of independent data items, and
the parameterization includes
for each independent data item, an item-parametrization of the independent data item, and
one or more statistical properties of the plurality of independent data items.

9. The method according to claim 1, further comprising:
generating a modified parameterization based on the parameterization; and wherein
the synthetic representation is generated based on the modified parameterization.

10. The method according to claim 1, wherein the local data comprises protected information, and the parameterization does not comprise the protected information.

11. The method according to claim 1, wherein
the machine learned model is an image processing function configured to generate an image processing result based on image data,
the local data includes training image data,
the parameterization includes a parameterization of the training image data, and
the synthetic representation includes synthetic image data generated by the generative AI function based on the parameterization of the training image data.

12. The method according to claim 11, wherein
the machine learned model is configured to generate the image processing result based on medical image data, the image processing result being selected from
a detection result of a medical finding in the medical image data,
a classification of a medical finding in the medical image data, or
a segmentation of the medical image data, and the training image data includes medical image data.

13. The method according to claim 1, further comprising:
providing the updated machine learned model to a second local site that is different from the first local site.

14. A model aggregator device for federated learning of a machine learned model, the model aggregator device comprising:
an interface unit configured to
receive, from a first local site that is remote from the model aggregator device, a local update of the machine learned model and a parameterization of local data, wherein the local update was generated at the first local site based on the local data; and
a computing unit configured to
generate a synthetic representation of the local data based on the parameterization using a generative AI function,
evaluate the local update using the synthetic representation to obtain an evaluation result indicative of performance of the local update, and
update the machine learned model based on the evaluation result and the local update.

15. A non-transitory computer program product comprising program elements that induce a computing unit of a model aggregator device for federated learning of a machine learned model to perform the method according to claim 1 when the program elements are loaded into a memory of the computing unit.

16. A non-transitory computer-readable medium on which program elements are stored, the program elements being readable and executable by a computing unit of a model aggregator device for federated learning of a machine learned model to cause the model aggregator device to perform the method according to claim 1 when the program elements are executed by the computing unit.

17. The method according to claim 10, wherein the protected information is protected personal information.

18. The method according to claim 2, wherein
the generative AI function is configured to generate the synthetic representation based on a natural language prompt indicating the synthetic representation to be generated, and
the generating includes
obtaining the natural language prompt based on the parameterization, and
inputting the natural language prompt into the generative AI function to generate the synthetic representation.

19. The method according to claim 18, further comprising:
adding the synthetic representation to an existing test data set accessible for the model aggregator device for at least one of validating or testing the machine learned model to generate an extended test data set, and wherein
the evaluating evaluates the local update based on the extended test data set.

20. The method according to claim 6, wherein the determining a data quality of the synthetic representation comprises:
generating a natural language summary based on the synthetic representation;
comparing the natural language summary to a natural language prompt indicating the synthetic representation; and
determining the data quality based on the comparing the natural language summary to the natural language prompt.
